(19) United States
(12) Patent Application Publication
(10) Pub. No.: US 2026/0314940 A1
(43) Pub. Date: Oct. 15, 2026

(54) DIAGNOSTIC DEVICE FOR ASSESSING THE ENDOTHELIAL FUNCTION OF A HUMAN SUBJECT

(71) Applicant: STRUMEDICAL S.R.L., MONTECASSIANO (IT)

(72) Inventors: Chiara CALAMANTI, MONTECASSIANO (IT); Mario RABUINI, MONTECASSIANO (IT)

(21) Appl. No.: 19/108,459

(22) PCT Filed: Sep. 5, 2023
(86) PCT No.: PCT/IB2023/058777

§ 371 Date: Mar. 4, 2025

(30) Foreign Application Priority Data
Sep. 6, 2022 (IT) 102022000018207

(51) Int. Cl. A61B 5/02; A61B 5/00

(52) U.S. Cl. CPC A61B 5/02007; A61B 5/7221

(57) ABSTRACT

A diagnostic device for assessing the endothelial function of a subject, having a sphygmomanometric sleeve applicable to a forearm of the subject and provided with a pressure adjustment unit, two photoplethysmographic sensors applicable to a finger of the hand of the forearm and a finger of the other hand, a measuring unit for controlling the pressure adjustment unit and the photoplethysmographic sensors and for acquiring, by means of the latter, two signals relative to the two fingers, and a processing and control unit configured to control the measuring unit according to the two signals so as to execute a cycle comprising a calibration phase of the photoplethysmographic sensors, a basal phase, an occlusion phase of the brachial artery of the forearm, and a post-occlusion phase, and configured to filter and process the two signals so as to determine at least one endothelial function index.

### CROSS-REFERENCE TO RELATED APPLICATIONS

This patent application claims priority from Italian Patent Application No. 102022000018207 filed on Sep. 6, 2022, the entire disclosure of which is incorporated herein by reference.

### TECHNICAL FIELD

The present invention relates to a diagnostic device for assessing the endothelial function of a human subject.

### BACKGROUND

The endothelium is the largest organ of the human body and covers all the vessels of the cardiovascular system (heart, arteries, veins, capillaries and lymphatic system). Considered as simple tissue in the past, on the contrary the endothelium carries out a role of primary importance in the modulation of the blood vessel tone and of the hematic flow, besides other numerous functions such as the regulation of the inflammatory and immune processes, the vascular permeability, etc. A damage to the endothelium entails a pathological condition characterized by a reduced vasodilatation, contributing towards various cardiovascular alterations such as atherosclerosis, hypertension and thrombosis.

To date, the most widespread method for assessing the endothelium is measuring, by means of ultrasound examination, the diameter of the brachial artery under basal conditions, subsequently inducing a brief ischemia in a forearm of the subject and then measuring the variations of the diameter in the brachial artery after the ischemia.

The aforementioned method for assessing the endothelium has the advantage of not being invasive, but unfortunately, since it is an ultrasound examination, it strongly depends on the ability of the doctor performing it and thus has a reduced inter- and intra-operator reproducibility.

In order to try increasing the reproducibility, a mechanical arm having a micrometric adjustment so as to prevent the vascular probe from moving and a specific software for measuring the calibre variations in the artery are often utilized. In other words, in the clinical practice the aforementioned method is laborious to perform and therefore not very practicable. Hence, such method is mostly utilized within the scope of clinical investigation, i.e. does not fall within the daily hospital practice, let alone in the outpatient practice.

### SUMMARY

The object of the present invention is to provide an instrument which allows assessing the endothelial function of a human subject, which is exempt from the above-described drawbacks and, at the same time, is cost-effective to manufacture, easy to utilize and especially provides results substantially independent of the operator using it.

In accordance with the present invention, a diagnostic device for assessing the endothelial function of a subject is provided, in accordance with the appended claims.

### BRIEF DESCRIPTION OF THE DRAWINGS

The present invention will now be described with reference to the accompanying drawings, which illustrate a non-limiting example embodiment thereof, wherein:

FIG. 1 illustrates the diagnostic device of the present invention for assessing the endothelial function of a human subject;

FIG. 2 illustrates the diagnostic device of FIG. 1 when in use on the human subject;

FIG. 3 illustrates a block diagram of internal parts of the diagnostic device of FIG. 1;

FIG. 4 illustrates a simplified block diagram of a part of the software architecture of a unit of the diagram of FIG. 3;

FIG. 5 illustrates a simplified flowchart relative to a measuring cycle executed by the diagnostic device of FIG. 1;

FIG. 6 illustrates an example of the two filtered signals derived from the signals acquired by two sensors of the diagnostic device of FIG. 1 during the measuring cycle of FIG. 5, with reference to the calculation of an endothelial function index;

FIGS. 7A and 7B illustrate two examples of one of the two filtered signals of FIG. 6 obtained from a healthy human subject and from a subject having an endothelial dysfunction, respectively; and

FIG. 8 illustrates another example of one of the two filtered signals of FIG. 6 with reference to the calculation of a further endothelial function index; and

FIG. 9 illustrates a waveform of a heartbeat part of one of the two filtered signals of FIG. 6, on which some points of reference are illustrated.

### DESCRIPTION OF EMBODIMENTS

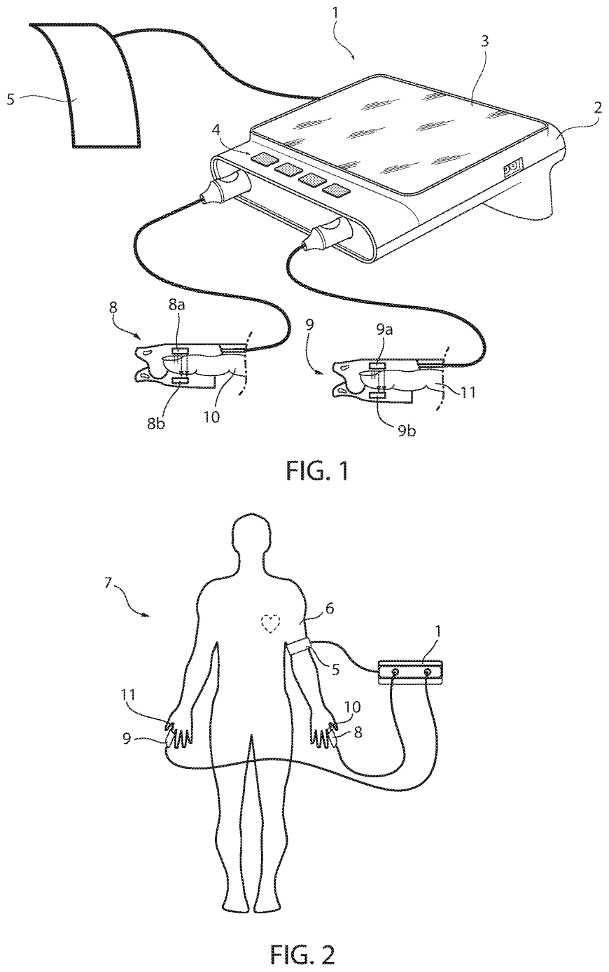

In FIGS. 1 and 2, reference numeral 1 generically indicates, as a whole, the diagnostic device of the present invention. The diagnostic device 1 comprises a box-shaped body 2, a display 3, preferably of touch-screen type, facing from an upper side of the box-shaped body 2, a push-button interface 4 for enabling an operator to input basic commands, a sphygmomanometric sleeve 5 applicable to a forearm 6 (FIG. 2) of a subject 7 and two photoplethysmographic sensors 8 and 9 applicable to two respective fingers of two different hands of the subject 7. In particular, as is illustrated in FIG. 2, the photoplethysmographic sensor 8 is applied to a finger 10, for example the index finger, of the hand of the forearm 6 and the other photoplethysmographic sensor 9 is applied to a finger 11, for example the index finger, of the other hand of the subject 7.

Each photoplethysmographic sensor 8, 9 comprises a source of light or infrared radiation 8a, 9a adapted to light a portion of skin of the finger 10, 11 and a receiver 8b, 8b which detects the variations of light absorbed by the portion of skin, such variations being due to the blood perfusion in the dermis and in the subcutaneous regions of the portion of skin. The photoplethysmographic sensors 8 and 9 are of known type and thus will not be described in further detail.

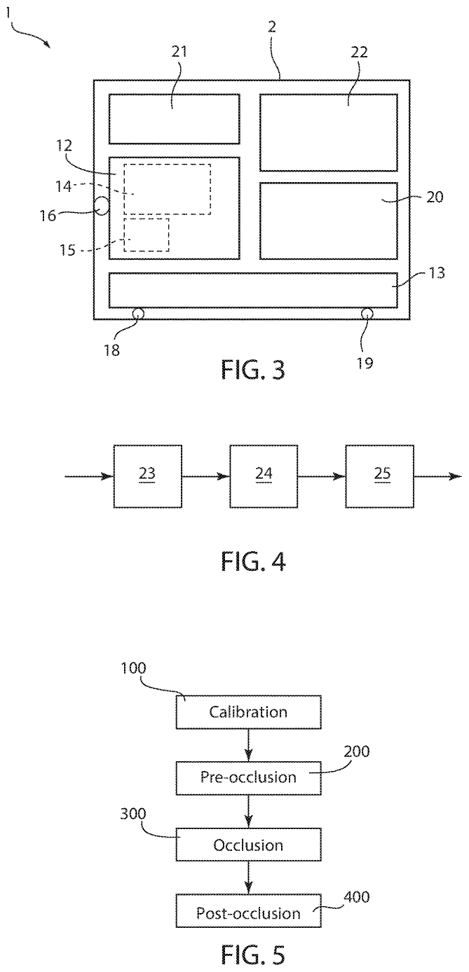

With reference to FIG. 3, the diagnostic device 1 comprises, inside the box-shaped body 2, a pressure adjustment unit 12 for adjusting the pressure of the sphygmomanometric sleeve 5 and a measuring unit 13 for controlling the pressure adjustment unit 12 and the photoplethysmographic sensors 8 and 9 and for acquiring, by means of the latter, a first signal S1 relative to the finger 10 and a second signal S2 relative to the other finger 11. The pressure adjustment unit 12 comprises a pump 14, an outlet valve 15, and a door 16 for the pneumatic connection of the sphygmomanometric sleeve 5. The measuring unit 13 comprises two doors 18 and 19 for the electrical connection of the photoplethysmographic sensors 8 and 9.

The diagnostic device 1 comprises, still inside the box-shaped body 2, a processing and control unit 20, a battery pack 21 for providing electric energy, and a power supply management unit 22, which manages the power supply of all the electrical and electronic parts of the diagnostic device 1.

The processing and control unit 20 is configured to control the measuring unit 13 so as to execute, in use, a measuring cycle which leads to the determination of one or more endothelial function indexes and, optionally, of heartbeat parameters which can be utilized for studying the severity of a possible endothelial dysfunction, as it will be specifically explained in the following of the present document. The measuring cycle is executed in an automatic manner.

With reference to FIG. 4, the processing and control unit 20 comprises at least one pre-processing module, and in particular three pre-processing modules indicated by 23, 24 and 25, for filtering the two signals S1 and S2 so as to obtain two respective filtered signals SF1 and SF2. The processing and control unit 20 is configured to determine the aforementioned endothelial function indexes and heartbeat parameters on the basis of the filtered signals SF1 and SF2.

Specifically, said three pre-processing modules comprise in cascade, in the following order, a low-pass filter 23, an artefact removal module 24 and an anomalous values removal module 25. The modules 23, 24 and 25 process in an identical and mutually independent manner the two signals S1 and S2. In other words, the functions of the modules 23, 24 and 25 are duplicated for the two signals S1 and S2.

The low-pass filter 23 is adapted to remove the oscillations at high-frequencies, with respect to those of the heartbeat, generated by ambient electromagnetic disturbances, such as for example the electromagnetic field generated by the electric network which comprises a fundamental frequency at 50 Hz and harmonic oscillations at higher frequencies.

The artefact removal module 24 is adapted to remove a signal component due to the body movements of the subject 7, and in particular of the fingers 10 and 11 with respect to the photoplethysmographic sensors 8 and 9. For example, the artefact removal module 24 implements a procedure known in the electrocardiographic field as baseline wander filtering.

The anomalous values removal module 25 is adapted to remove the isolated peak values of the signal, which are also often due to the body movements of the subject. For example, the anomalous values removal module 25 implements a procedure similar to the one followed by the “isoulier” function by The MathWorks Inc.

The three pre-processing modules 23, 24 and 25 are implemented in hardware, or are implemented by a software. In the latter case, the software is stored in a memory of the processing and control unit 20 and is executed by a processor of the processing and control unit 20.

With reference to FIG. 5, the measuring cycle, which is controlled by the processing and control unit 20 so as to be executed automatically, comprises, in the following order, a calibration phase (step 100), in which the photoplethysmographic sensors 8 and 9 are calibrated on the fingers 10 and 11 and the sphygmomanometric sleeve 5 is left deflated, a basal or pre-occlusion phase (step 200), in which the signals S1 and S2 are acquired while the sphygmomanometric sleeve 5 is left deflated, an occlusion phase (step 300), in which the signals S1 and S2 are acquired and the sphygmomanometric sleeve 5 is rapidly inflated, kept inflated at a maximum pressure value Pmax such to occlude the brachial artery of the forearm 6 and determined according to the signal S1, and then rapidly deflated, and a post-occlusion phase (step 400), in which the signals S1 and S2 are acquired while the sphygmomanometric sleeve 5 is left deflated. In the following, for simplicity, the phases 200, 300 and 400 are also called as a whole measurement phases.

With regard to the calibration phase, the processing and control unit 20 is configured to control the measuring unit 13 so that the latter measures, on the basis of the signals S1 and S2, a perfusion index for each of the photoplethysmographic sensors 8 and 9 and verifies that it is greater than a threshold value. The measurement of the perfusion index is known. Furthermore, the measuring unit 13 adjusts the intensity of the sources 8a and 9a until the receivers 8b and 9b detect respective light signals above a certain light threshold value. This enables adapting the operation of the photoplethysmographic sensors 8 and 9 to the characteristics of the tissue of the fingers 10 and 11 and to the ambient light.

At the beginning of the occlusion phase, the sphygmomanometric sleeve 5 is rapidly inflated to a pressure value P1 corresponding to a substantially null value of the signal S1 and at the end of the occlusion phase the sphygmomanometric sleeve 5 is rapidly deflated. When the signal S1 is close to the null value, it means that the blood flow in the finger 10 is substantially null, and thus that the brachial artery of the forearm 6 is actually occluded. The maximum pressure value Pmax is determined according to the pressure value P1.

Advantageously, the maximum pressure value Pmax is given by the sum of the pressure value P1 with a predetermined pressure increase ΔP. This allows preventing the pressure from not being sufficient for occluding the brachial artery due to muscle spasms or movements of the forearm 6. For example, the pressure increase ΔP is between 30 and 50 mmHg, preferably it is equal to 40 mmHg. The temporary occlusion of the brachial artery induces a temporary ischemia at the level of the forearm 6.

The calibration phase has a duration T1 between 1 and 5 minutes, preferably equal to 3 minutes. The pre-occlusion phase, the occlusion phase and the post-occlusion phase have respective durations T2, T3, T4, each between 3 and 6 minutes, preferably equal to 5 minutes.

The durations T1, T2, T3, T4 are singularly pre-settable by the operator, depending on the investigations which have to be carried out. In accordance with an embodiment, the duration T1 is equal to 3 minutes and the durations T2, T3 and T4 are all three equal to 5 minutes. In accordance with a further embodiment, the duration T1 is equal to 3 minutes, the duration T2 is equal to 3 minutes, the duration T3 is equal to 4 minutes and the duration T4 is equal to 3 minutes.

The inflation of the sphygmomanometric sleeve 5 to the maximum pressure value Pmax is an initial transitory sub-phase of the occlusion phase which has a duration between 30 and 40 seconds.

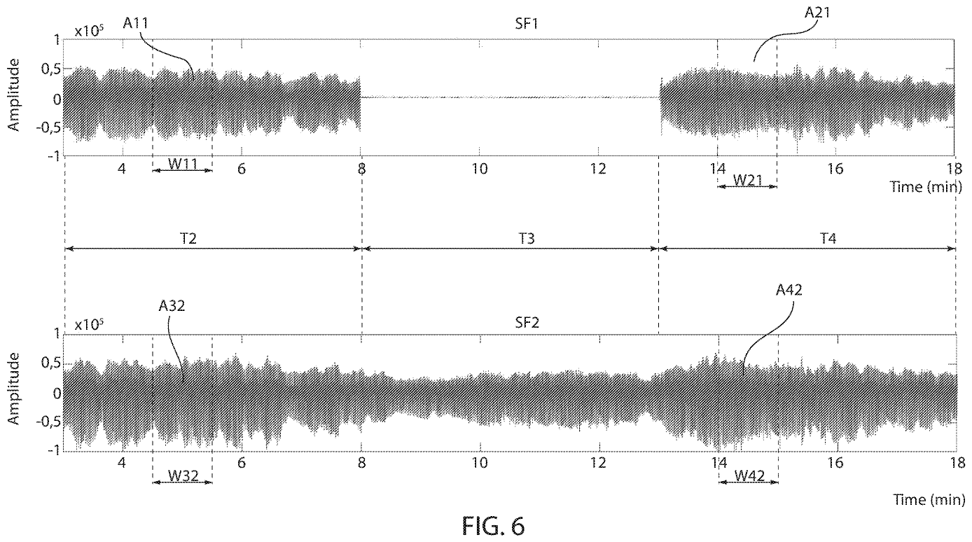

With reference to FIG. 6, which illustrates an example of the filtered signals SF1 and SF2 during the measurement phases 200, 300, 400, the processing and control unit 20 is configured to determine a first endothelial function index, indicated by EI, in the following manner.

Four areas subtended by the envelopes of the filtered signals SF1 and SF2 within four respective time windows are calculated. In particular, these four areas are:
a first area A11 subtended by the envelope of the filtered signal SF1 within a first time window W11 of the pre-occlusion phase, the latter extending according to a time interval having the duration T2;
a second area A21 subtended by the envelope of the filtered signal SF1 within a second time window W21 of the post-occlusion phase, the latter extending according to a time interval having the duration T4;
a third area A32 subtended by the envelope of the filtered signal SF2 within a third time window W32 of the pre-occlusion phase; and
a fourth area A42 subtended by the envelope of the filtered signal SF2 within a fourth time window W42 of the post-occlusion phase.

The endothelial function index EI is calculated as the product of a ratio of the area A21 to the area A11 by a ratio of the area A32 to the area A42, i.e. with the following formula:

$$IE = \frac{A 21}{A 11} \cdot \frac{A 32}{A 42} .$$

The time windows W11, W21, W32 and W42 have respective durations, each between 0.5 and 1.5 minutes, preferably equal to 1 minute. The durations of the windows W11, W21, W32 and W42 can be set at the discretion of the operator. The location of the time windows W11 and W32 within the pre-occlusion phase, i.e. within the interval having duration T2, and of the time window W42 within the post-occlusion phase, i.e. within the interval having duration T4, is not relevant. The time window W21 begins after a predetermined delay D from the end of the occlusion phase, i.e. at the end of the duration T3. The delay D is between 0.5 and 1.5 minutes, preferably equal to 1 minute. However, also the precise location of the windows W11, W21, W32 and W42 within the relative pre-occlusion and post-occlusion phases is settable at the discretion of the operator.

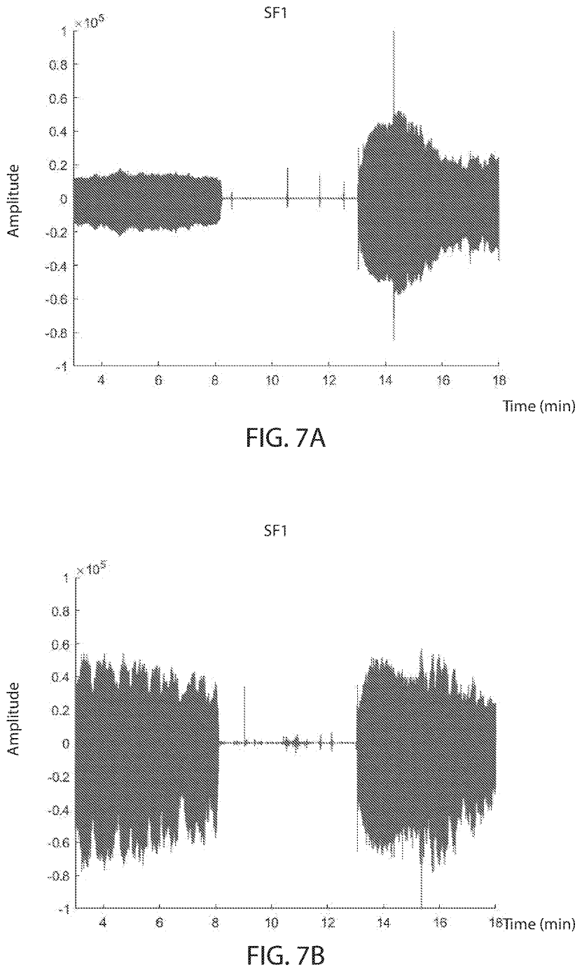

The endothelial function index EI was assessed with a clinical investigation, which highlighted the existence of a threshold value which allows providing indications on the state of the subject 7. FIG. 7A illustrates the course of the filtered signal SF1 of a healthy subject, to which an endothelial function index EI greater than said threshold value corresponds, and FIG. 7B illustrates the course of the filtered signal SF1 of a subject having an endothelial dysfunction, to which an endothelial function index EI lesser than said threshold value corresponds.

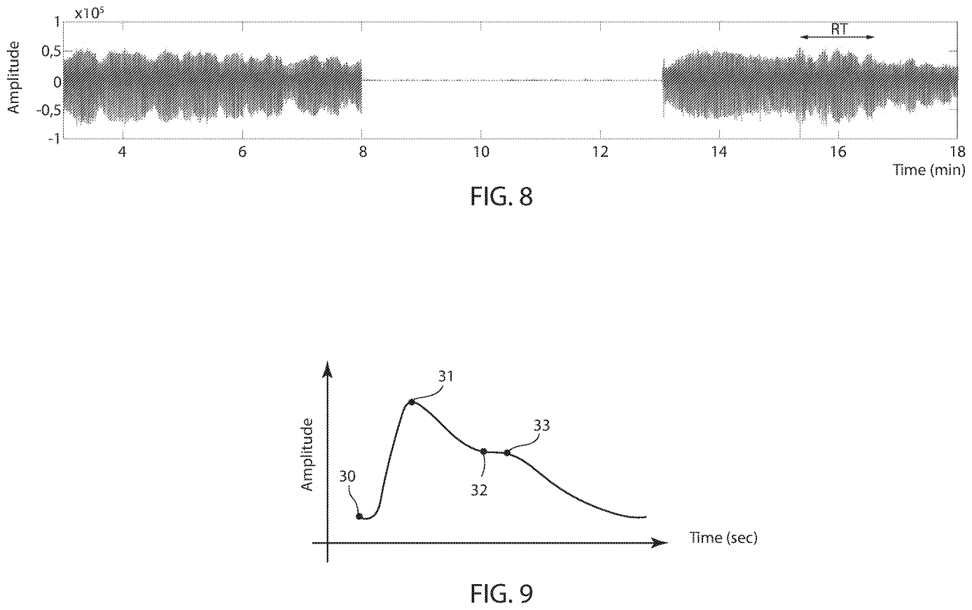

With reference to FIG. 8, which illustrates another example of the filtered signal SF1 during the measurement phases 200, 300, 400, the processing and control unit 20 is configured to determine a further endothelial function index, indicated by RT and called in the following recovery time.

An average amplitude of the filtered signal SF1 is calculated during the post-occlusion phase. The recovery time RT is calculated as that time interval in the post-occlusion phase between the instant in which the filtered signal SF1 reaches the maximum amplitude and the instant in which the filtered signal SF1 reaches said average amplitude.

The higher the recovery time RT, the greater the endothelial dysfunction. This can be explained by the fact that a high value of recovery time RT expresses a poor recovery capacity of the endothelium following the temporary ischemia caused by the occlusion phase, and the poor recovery capacity is necessarily linked to a reduced endothelial function. The combined reading of the value of the recovery time RT and of the endothelial function index could provide an indication of how serious a pathology linked to the endothelial dysfunction is.

The numerical values of the endothelial function index EI and of the recovery time RT can be displayed directly on the display 3 of the diagnostic device 1.

Optionally, the processing and control unit 20 is configured to process the filtered signal SF1 in the pre-occlusion phase or/and in the post-occlusion phase so as to extract one or more heartbeat parameters in the manner described in the following.

The filtered signal SF1 is composed of a sequence of a high number of heartbeat waveforms, each of which is similar to the example illustrated in FIG. 9. With reference to FIG. 9, each heartbeat waveform is characterized by points of reference, known in the electrocardiographic and photoplethysmographic fields, such as for example the dicrotic minimum 30, the systolic peak 31, the dicrotic notch 32 and the diastolic peak 33, and by heartbeat parameters also known in the electrocardiographic field and calculable on the basis of the aforementioned points of reference of one waveform or of two contiguous waveforms. In particular, the aforementioned heartbeat parameters are known in the literature as:
Systolic Amplitude,
Inflection Point Area,
Pulse Interval,
Heart Rate,
Peak-to-Peak Time,
Stiffness Index,
Augmentation Index.

The heartbeat parameters which are determined by the processing and control unit 20 are thus selected from a group comprising Systolic Amplitude, Inflection Point Area, Pulse Interval, Heart Rate, Peak-to-Peak Time, Stiffness Index, Augmentation Index.

However, the aforementioned heartbeat parameters are defined on a single heartbeat waveform.

With the purpose of associating at least one heartbeat parameter with the filtered signal SF1, from the latter a predetermined number of signal portions selected corresponding to the waveforms of as many random heartbeats, in the pre-occlusion phase or/and in the post-occlusion phase. In other words, the signal portions corresponding to as many heartbeats are randomly selected along the overall waveform of the filtered signal SF1 in the pre-occlusion phase or/and in the post-occlusion phase. For each of the selected heartbeats, a value of at least one heartbeat parameter is calculated on the basis of the waveform of the heartbeat and then an average value of said heartbeat parameter is calculated among all the values of the selected heartbeats.

In particular, for each selected heartbeat, the calculation of the value of the heartbeat parameter is preceded by the identification of the points of reference 30-33 of the waveform of the heartbeat. The points of reference 30-33 are identified by processing first derivative and second derivative of the waveform of the heartbeat. The value of the heartbeat parameter is calculated on the basis of the points of reference 30-33 of the relative waveform by means of simple mathematical relationships. For example, the systolic amplitude is equal to the difference of the amplitude values of the systolic peak 31 and of the dicrotic minimum 30 and the peak-to-peak time is equal to the difference of the time instants of the systolic peak 31 and of the diastolic peak 33.

The above-described measuring cycle, with reference to FIG. 5, and the calculation of the endothelial function indexes EI, RT and of the heartbeat parameters on the basis of the filtered signals SF1 and SF2 are implemented by a software which is stored in the memory of the processing and control unit 20 and is executed by the processor of the processing and control unit 20.

The heartbeat parameters allow refining a diagnosis based on the two endothelial function indexes EI and RT. Advantageously, the processing and control unit 20 is configured to execute a machine learning algorithm which allows classifying the severity of an endothelial dysfunction in correlation with one or more heartbeat parameters.

The main advantage of the above-described diagnostic device 1 is to enable a diagnosis of the endothelium in an automatic manner and without the need for the intervention of a specialized doctor in the utilization of complex medical instruments. The diagnosis can be synthetically expressed on the display by means of numerical values of one or more suitably calculated endothelial function indexes. Furthermore, the diagnostic device 1 is relatively cost-effective to manufacture, as it comprises widespread components, such as the sphygmomanometric sleeve 5, a pressure adjustment unit 12 comprising a pump 14 and an outlet valve 15, and two photoplethysmographic sensors 8 and 9.

1. A diagnostic device for assessing the endothelial function of a subject, the diagnostic device comprising a sphygmomanometric sleeve applicable to a first forearm of the subject, a pressure adjustment unit for adjusting the pressure of the sphygmomanometric sleeve, two photoplethysmographic sensors applicable to a first finger of the hand associated with the first forearm and, respectively, to a second finger of the other hand of the subject, a measuring unit for controlling the pressure adjustment unit and the photoplethysmographic sensors and for acquiring, by means of the latter, a first signal (S1) relative to the first finger and a second signal (S2) relative to the second finger, and a processing and control unit, which is configured to control the measuring unit so as to execute, in use, a cycle comprising, in the following order, a first phase, in which the photoplethysmographic sensors are calibrated on the first finger and second finger, a second phase, in which the sphygmomanometric sleeve is left deflated, a third phase, in which the sphygmomanometric sleeve is kept inflated to a maximum pressure value (Pmax) determined according to the first signal (S1), and a fourth phase, in which the sphygmomanometric sleeve is left deflated; the processing and control unit comprising at least one pre-processing module for filtering first and second signals (S1, S2) to obtain a first and second filtered signal (SF1, SF2) and being configured to determine at least one endothelial function index (EI, RT) on the basis of at least the first filtered signal (SF1) in the second phase and the fourth phase.

2. The diagnostic device according to claim 1, wherein the processing and control unit is configured to control the measuring unit such that at the beginning of the third phase the sphygmomanometric sleeve is rapidly inflated to a first pressure value (P1) corresponding to a substantially null value of the first signal (S1) and at the end of the third phase the sphygmomanometric sleeve is rapidly deflated; the maximum pressure value (Pmax) being determined as a function of the first pressure value (P1).

3. The diagnostic device according to claim 1, wherein said first phase has a duration (T1) between 1 and 5 minutes.

4. The diagnostic device according to claim 1, wherein said second phase, third phase and fourth phase have respective durations (T2-T4), each between 3 and 6 minutes.

5. The diagnostic device according to claim 1, wherein said processing and control unit is configured to calculate a first area (A11) and a second area (A21) subtended by the envelope of the first filtered signal (SF1) within a first time window (W11) of the second phase and, respectively, a second time window (W21) of the fourth phase, a third area (A32) and a fourth area (A42) subtended by the envelope of the second filtered signal (SF2) within a third time window (W32) of the second phase and, respectively, a fourth time window (W42) of the fourth phase, and a first endothelial function index (EI) as the product of a ratio of second area (A21) to first area (A11) by a ratio of third area (A32) to fourth area (A42).

6. The diagnostic device according to claim 5, wherein said first, second, third and fourth time windows (W11, W21, W32, W42) have respective durations, each between 0.5 and 1.5 minutes.

7. The diagnostic device according to claim 5, wherein said second time window (W21) begins after a predetermined delay (D) from the end of the third phase, said delay (D) being between 0.5 and 1.5 minutes.

8. The diagnostic device according to claim 1, wherein said processing and control unit is configured to calculate an average amplitude of the first filtered signal (SF1) during the second phase and a second endothelial function index (RT) as that time interval in the fourth phase between the instant in which the first filtered signal (SF1) reaches the maximum amplitude and the instant in which the first filtered signal (SF1) reaches said average amplitude.

9. The diagnostic device according to claim 1, wherein the processing and control unit is configured to select, from the first filtered signal (SF1) in the second phase or/and in the fourth phase, a predetermined number of signal portions corresponding to the waveforms of as many random heartbeats, to calculate, for each of said heartbeats, a value of at least one heartbeat parameter on the basis of its waveform, and to calculate the average value of said heartbeat parameter among all the values of said heartbeats.

10. The diagnostic device according to claim 1, wherein each of the photoplethysmographic sensors comprises a source of light or infrared radiation and a corresponding receiver and the processing and control unit is configured to control the measuring unit such that the latter, during said first phase, measures and verifies the amplitude of a perfusion index for both photoplethysmographic sensors on the basis of the first and second signals (S1, S2), and adjusts the intensity of the radiation emitted by the radiation sources to adapt the operation of the photoplethysmographic sensors to the characteristics of the finger tissue and the ambient light.

11. The diagnostic device according to claim 1, wherein said at least one pre-processing module comprises a low-pass filter and at least an artefact removal module, the latter to remove artefacts on the first and second signals (S1, S2) due to movements of the first and second fingers with respect to the photoplethysmographic sensors.

12. The diagnostic device according to claim 1, wherein the first phase (T1) has a duration equal to 3 minutes.

13. The diagnostic device according to claim 1, wherein the second phase, the third phase and the fourth phase have respective durations (T2-T4) equal to 5 minutes.

14. The diagnostic device according to claim 5, wherein the first, second, third and fourth time windows (W11, W21, W32, W42) have respective durations equal to 1 minute.

15. The diagnostic device according to claim 7, wherein the delay (D) is equal to 1 minute.
